(19) United States
(12) Patent Application Publication
(10) Pub. No.: US 2025/0017570 A1
(43) Pub. Date: Jan. 16, 2025

(54) ULTRASOUND DIAGNOSTIC APPARATUS AND MODEL OPERATION VERIFICATION METHOD

(71) Applicant: FUJIFILM Healthcare Corporation, Chiba (JP)

(72) Inventors: TOMOFUMI NISHIURA, Chiba (JP); TSUYOSHI MATSUMOTO, Tokyo (JP)

(73) Assignee: FUJIFILM Healthcare Corporation, Chiba (JP)

(21) Appl. No.: 18/765,333

(22) Filed: Jul. 8, 2024

(30) Foreign Application Priority Data
Jul. 12, 2023 (JP) 2023-114244

(51) Int. Cl. A61B 8/08; A61B 8/00; G16H 15/00

(52) U.S. Cl. CPC A61B 8/5207; A61B 8/463; G16H 15/00

(57) ABSTRACT

A log management unit creates a log in which an operation of an ultrasound diagnostic apparatus is recorded. A model operation management unit saves an analysis target image input to an image analysis model and an analysis result of the image analysis model for each operation of the image analysis model. In a case where a specific model operation record in the log is selected, the report creation unit creates a model operation report. The model operation report includes the analysis target image input to the image analysis model and the analysis result of the image analysis model.

### CROSS-REFERENCE TO RELATED APPLICATION

This application is based on and claims priority under 35 USC 119 from Japanese Patent Application No. 2023-114244 filed Jul. 12, 2023.

### BACKGROUND OF THE INVENTION

### 1. Field of the Invention

The present disclosure relates to an ultrasound diagnostic apparatus and a model operation verification method, and more particularly, to a technique for verifying an operation of an image analysis model that is artificial intelligence (AI).

### 2. Description of the Related Art

An ultrasound diagnostic apparatus is used in an ultrasound examination of a living body. The ultrasound diagnostic apparatus is a medical apparatus that generates and displays an ultrasound image based on a reception signal obtained by transmitting and receiving ultrasound waves. A tomographic image is known as a representative ultrasound image.

In recent years, an ultrasound diagnostic apparatus having an image analysis model generated through machine learning has been increasingly prevalent. Ultrasound diagnostic apparatuses described in JP6423540B and JP2020-204970A include an image analysis model that automatically identifies (or classifies) a tissue cross section based on a tomographic image. Examples of other image analysis models include a model that detects a lesion part included in the tomographic image, and a model that measures a tissue on the tomographic image.

Some ultrasound diagnostic apparatuses have a log creation function. In such an ultrasound diagnostic apparatus, a log is created by sequentially recording a plurality of operations in time series order (refer to JP2021-507325A). A series of past operations can be verified by referring to the log. Specifically, it is possible to refer to the log to confirm that an operation of each individual component is appropriate or to specify a malfunction in any component. In general, the log is composed of a plurality of records corresponding to a plurality of operations (including event occurrence and processing execution) that have occurred in time series order. Information included in each record is finite and generally simple. In each record in the log, detailed information is not linked.

### SUMMARY OF THE INVENTION

Performance of the image analysis model is decreased because of various factors such as a decrease in quality of an analysis target image and insufficient machine learning. It is desired to periodically verify the performance or the state of the image analysis model. Conventional ultrasound diagnostic apparatuses do not sufficiently meet such a demand. For example, in the ultrasound diagnostic apparatus having the log creation function, the operation of the image analysis model is recorded on the log. However, the content of each record in the log is finite, and the content of each record is generally simple. It is difficult to verify the operation of the image analysis model in detail by referring to each record.

An object of the present disclosure is to support verification of an operation of an image analysis model. Alternatively, an object of the present disclosure is to support detailed verification of the operation of the image analysis model.

According to the present disclosure, there is provided an ultrasound diagnostic apparatus comprising: an analysis unit that includes an image analysis model which has been trained through machine learning to analyze an ultrasound image; a log creation unit that creates a log consisting of a plurality of operation records by recording a plurality of operations including an operation of the image analysis model in time series order; a saving unit that saves detailed information for model operation verification for each operation of the image analysis model; and a report creation unit that creates, in a case where a specific model operation record in the log is selected, a model operation report including detailed information associated with the specific model operation record.

According to the present disclosure, there is provided a model operation verification method comprising: a step of creating a log consisting of a plurality of operation records by recording, in time series order, a plurality of operations including an operation of an image analysis model that has been trained through machine learning to analyze an ultrasound image; a step of saving detailed information for model operation verification for each operation of the image analysis model; a step of displaying the log; a step of accepting selection of a specific model operation record in the displayed log; a step of specifying detailed information associated with the specific model operation record; a step of creating a model operation report including the detailed information; and a step of displaying the model operation report.

According to the present disclosure, it is possible to support the verification of the operation of the image analysis model. Alternatively, according to the present disclosure, it is possible to support detailed verification of the operation of the image analysis model.

### BRIEF DESCRIPTION OF THE DRAWINGS

FIG. 1 is a block diagram showing a configuration example of an ultrasound diagnostic apparatus according to an embodiment.

FIG. 2 is a block diagram showing a configuration example of a model operation management unit.

FIG. 3 is a diagram showing a detailed record column.

FIG. 4 is a diagram showing an example of a log.

FIG. 5 is a diagram showing a first example of a model operation report.

FIG. 6 is a diagram showing a second example of the model operation report.

FIG. 7 is a diagram showing a third example of the model operation report.

FIG. 8 is a diagram showing a fourth example of the model operation report.

### DESCRIPTION OF THE PREFERRED EMBODIMENTS

Hereinafter, an embodiment will be described with reference to the drawings.

### (1) Outline of Embodiment

An ultrasound diagnostic apparatus according to the embodiment includes an analysis unit, a log creation unit, a saving unit, and a report creation unit. The analysis unit includes an image analysis model that has been trained through machine learning to analyze an ultrasound image. The log creation unit creates a log consisting of a plurality of operation records by recording a plurality of operations including an operation of the image analysis model in time series order. The saving unit saves detailed information for model operation verification for each operation of the image analysis model. The report creation unit creates, in a case where a specific model operation record in the log is selected, a model operation report including detailed information associated with the specific model operation record.

With the above-described configuration, the performance or the state of the image analysis model can be verified in detail by referring to the detailed information included in the model operation report. The detailed information is information that is more detailed than information included in the log. For example, the model operation record in the log includes information such as an execution time of the image analysis model and an identifier of the image analysis model. In a case where an amount of information thereof is defined as a first amount of information, the detailed information has a second amount of information larger than the first amount of information. The detailed information according to the embodiment includes at least one of an analysis target image input to the image analysis model or an analysis result of the image analysis model. In general, such information may be discarded, but in the embodiment, such information is saved as the detailed information for model operation verification. More specifically, the detailed information is saved while being associated with the model operation record in the log. Such association is not generally recognized in conventional general logs.

In the embodiment, the saving unit saves the analysis target image as the above-described detailed information for each operation of the image analysis model. The analysis target image is an ultrasound image input to the image analysis model or an image corresponding to the ultrasound image. The model operation report includes the analysis target image. With this configuration, it is possible to verify the operation of the image analysis model while referring to the analysis target image input to the image analysis model. All the ultrasound images input to the image analysis model may be saved as the analysis target image, or a part of all the ultrasound images input to the image analysis model may be saved as the analysis target image.

The ultrasound diagnostic apparatus according to the embodiment includes a conversion unit that converts the ultrasound image input to the image analysis model into a low-resolution image. The analysis target image is the low-resolution image. With this configuration, it is possible to reduce a storage capacity required to save the analysis target image. A saving period may be set for each analysis target image, and the analysis target image may be automatically erased upon the expiration of the saving period.

In the embodiment, the saving unit saves the analysis result of the image analysis model as the above-described detailed information for each operation of the image analysis model. The model operation report includes the analysis result. With this configuration, it is possible to verify the operation of the image analysis model while referring to the analysis result of the image analysis model. All the analysis results of the image analysis model may be saved, or a part of all the analysis results of the image analysis model may be saved.

In the embodiment, the above-described analysis result includes a plurality of scores corresponding to a plurality of classes. The report creation unit creates a graph based on the plurality of scores. The model operation report includes the graph. With this configuration, it is easy to verify the operation of the image analysis model.

In the embodiment, the saving unit includes an image saving unit and a record saving unit. The image saving unit saves the analysis target image for each operation of the image analysis model. The record saving unit saves a detailed record including the analysis result of the image analysis model for each operation of the image analysis model. The analysis target image is a low-resolution image generated from the ultrasound image input to the image analysis model. The detailed information in the model operation report includes the analysis target image, which is input information of the image analysis model, and the analysis result, which is output information of the image analysis model.

With the above-described configuration, it is possible to verify the operation of the image analysis model while referring to both the input information and the output information of the image analysis model. Therefore, it is possible to correctly specify the performance and the state of the image analysis model, and in a case where any malfunction occurs, it is possible to specify the cause of the malfunction. For example, in a case where it is found that the image analysis model is not functioning appropriately, the image analysis model may be retrained or the image analysis model may be replaced.

In the embodiment, each model operation record included in the log includes a portion embedded with a hyperlink for specifying a location of the detailed information associated with the model operation record. The model operation report is displayed through the operation on that portion. With this configuration, in a case where a concerning model operation record is discovered in a situation in which the log is referred to, it is possible to display the model operation report through a simple operation on that portion. In that case, the model operation report may be displayed in a pop-up manner. That is, the log and the model operation report may be displayed at the same time.

A model operation verification method according to the embodiment includes a log creation step, a saving step, a log display step, a selection step, a specification step, a report creation step, and a display step. In the log creation step, a plurality of operations including an operation of an image analysis model that has been trained through machine learning to analyze an ultrasound image are recorded in time series order, thereby creating a log consisting of a plurality of operation records. In the saving step, detailed information for model operation verification is saved for each operation of the image analysis model. In the log display step, the log is displayed. In the selection step, the selection of a specific model operation record in the displayed log is accepted. In the specification step, detailed information associated with the specific model operation record is specified. In the report creation step, a model operation report including the detailed information is created. In the display step, the model operation report is displayed.

A program for executing the above-described model operation verification method is installed in an information processing apparatus via a network or via a portable storage medium. The program is saved in a non-transitory storage medium in the information processing apparatus. The information processing apparatus is, for example, an ultrasound diagnostic apparatus.

### (2) Details of Embodiment

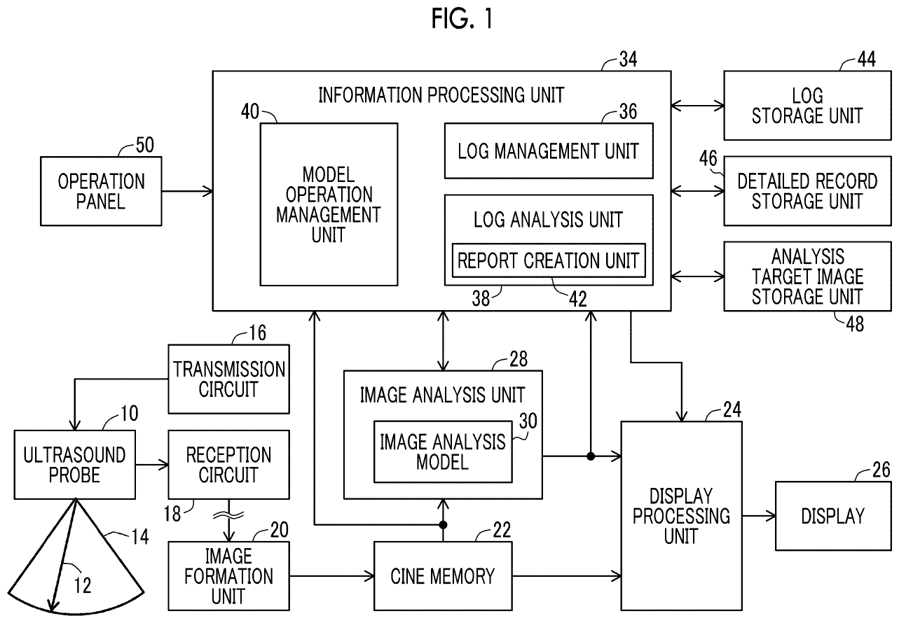

FIG. 1 shows the ultrasound diagnostic apparatus according to the embodiment. This ultrasound diagnostic apparatus is a medical apparatus that is installed in a medical institution, such as a hospital, and that is used in the ultrasound examination of the subject.

An ultrasound probe 10 is a device that transmits ultrasound waves into a living body and that receives reflected waves from the living body. The ultrasound probe 10 includes a transducer array consisting of a plurality of transducers. The transducer array forms an ultrasound beam 12. A beam scanning plane 14 is formed through electronic scanning of the ultrasound beam 12. The beam scanning plane 14 is repeatedly formed by repeating the electronic scanning with the ultrasound beam 12 in accordance with the transmission frame rate.

As an electronic scanning method of the ultrasound beam 12, an electronic linear scanning method, an electronic sector scanning method, and the like are known. A two-dimensional transducer array may be provided as the transducer array. Volume data can be acquired from a three-dimensional space in the living body by performing two-dimensional scanning with the ultrasound beam using the two-dimensional transducer array.

A transmission circuit 16 is an electronic circuit that functions as a transmission beam former, and outputs a plurality of transmission signals to the transducer array in parallel during transmission. As a result, a transmission beam is formed by the action of the transducer array.

A reception circuit 18 is an electronic circuit that functions as a reception beam former, and applies phase addition to a plurality of reception signals output in parallel from the transducer array during reception, thereby generating reception beam data. With the repetition of the electronic scanning, a reception frame data column is output from the reception circuit 18. Each piece of the reception frame data is composed of a plurality of pieces of reception beam data arranged in an electronic scanning direction. Each piece of the reception beam data is composed of a plurality of pieces of echo data arranged in a depth direction.

The reception frame data column is sent to an image formation unit 20 through a data processing unit (not shown). The data processing unit is a module that applies a plurality of kinds of processing to each individual piece of the reception beam data. The plurality of kinds of processing include logarithmic transformation, filtering, and the like.

The image formation unit 20 includes a digital scan converter (DSC). The DSC has a coordinate transformation function, a pixel interpolation function, and the like. A display frame data column is generated from the reception frame data column by the DSC. The display frame data column corresponds to a tomographic image as a moving image. Each individual piece of display frame data constituting the display frame data column corresponds to a tomographic image as a still image.

In the shown configuration example, the display frame data column is stored in a cine memory 22, and then the display frame data column read out from the cine memory 22 is sent to a display 26 via a display processing unit 24. The tomographic image as a moving image is displayed on the display 26, or the tomographic image as a still image is displayed on the display 26. The display processing unit 24 has an image combining function, a color processing function, and the like.

The above-described cine memory 22 is a temporary storage memory having a ring buffer structure. The cine memory 22 is configured with, for example, a semiconductor memory. The image formation unit 20 and the display processing unit 24 are each configured with, for example, a processor. The display 26 is configured with a liquid crystal display, an organic EL display device, or the like. In the image formation unit 20, an ultrasound image other than the tomographic image may be formed. For example, a blood flow image, an elasticity image, or the like may be formed.

An image analysis unit 28 includes an image analysis model 30 that is AI. The image analysis model 30 is a model that has been trained through machine learning, and is configured with, for example, a convolutional neural network (CNN). The image analysis model 30 according to the embodiment is a model that identifies a tissue cross section based on the tomographic image. Specifically, the image analysis model 30 is a model that performs class classification. As the image analysis model 30, a model that executes measurement on the tissue based on the tomographic image, a model that detects a lesion part included in the tomographic image, or the like may be used.

In the embodiment, the display frame data column is transferred from the cine memory 22 to the image analysis unit 28. The display frame data column is also transferred to an information processing unit 34. The image analysis model 30 executes image analysis on each piece of the display frame data constituting the display frame data column for each piece of the display frame data (that is, for each tomographic image) and outputs an analysis result for each piece of the display frame data. An analysis result column corresponding to the display frame data column is output from the image analysis unit 28 to the display processing unit 24 and an information processing unit 34. The analysis result column is sent to the display 26 via the display processing unit 24, and the analysis result column is displayed on the display 26. The image analysis unit 28 is configured with, for example, a processor. The image analysis unit 28 may analyze the reception frame data.

The information processing unit 34 is configured with, for example, a CPU that executes a program. The information processing unit 34 functions as a controller that controls an operation of each element constituting the ultrasound diagnostic apparatus. In FIG. 1, a plurality of functions exhibited by the information processing unit 34 are represented by a plurality of blocks. Specifically, the information processing unit 34 functions as a log management unit 36, a log analysis unit 38, and a model operation management unit 40. These will be described in detail below. An operation panel 50 is connected to the information processing unit 34. The operation panel 50 is an input device including a track ball, a plurality of switches, a plurality of knobs, and the like.

A log storage unit 44, a detailed record storage unit 46, and an analysis target image storage unit 48 are connected to the information processing unit 34. The storage units 44, 46, and 48 are configured with a semiconductor memory, a hard disk, or the like.

The log management unit 36 functions as a log creation unit and a log readout unit. Specifically, a plurality of operations executed by the ultrasound diagnostic apparatus are recorded in time series order, thereby creating a log consisting of a plurality of records. Typically, one record corresponds to one operation. The plurality of operations executed by the ultrasound diagnostic apparatus include a plurality of operations of the image analysis model. A plurality of records constituting the log include a plurality of records (a plurality of model operation records) corresponding to a plurality of model operations. Actually, the log management unit 36 registers the plurality of records in the log storage unit 44 in time series order. As a result, the log is created on the log storage unit 44.

In a case of referring to the log, the log management unit 36 reads out the plurality of records registered in the log storage unit 44, and the plurality of records are transferred to the display processing unit 24. Consequently, the records are displayed on the display as the log. A condition to be displayed is set by a user. For example, a display period is designated by the user. Alternatively, a display content is changed in accordance with a scroll operation of the user.

The log analysis unit 38 analyzes the content of the log. For example, the log analysis unit 38 has an aggregation function, a retrieval function, and the like. The log analysis unit 38 may calculate a usage rate for each image analysis model or may specify an operation (for example, input of approval or correction) related to the operation of the image analysis model. In addition, the log analysis unit 38 may calculate an examination time, a processing speed, and the like. The log analysis unit 38 according to the embodiment includes a report creation unit 42. This will be described below.

The model operation management unit 40 functions as detailed information saving means. That is, the model operation management unit 40 saves the ultrasound image (specifically, the tomographic image) input to the image analysis model 30 as the analysis target image for each operation of the image analysis model 30 and saves the analysis result (specifically, the plurality of scores) of the image analysis model 30. The analysis target image and the analysis result are basically information not included in the log and are detailed information for model operation verification.

More specifically, the model operation management unit 40 generates a detailed record for each operation of the image analysis model 30. The detailed record includes link information indicating a location of the analysis target image and a numerical column as the analysis result.

A plurality of detailed records are stored in the detailed record storage unit 46. A detailed record column is composed of the plurality of detailed records. In a case where the log stored in the log storage unit 44 is represented as a main log or a general-purpose log, the detailed record column stored in the detailed record storage unit 46 can be referred to as a sub-log or an image analysis model-dedicated log.

The analysis target image storage unit 48 stores an analysis target image column consisting of a plurality of analysis target images. As will be described below, in the embodiment, each individual analysis target image is a low-resolution image generated from the ultrasound image. The detailed record storage unit 46 and the analysis target image storage unit 48 may be integrated. For example, the analysis target image may be included in each individual detailed record.

As will be described below, an ID number for identifying the operation is issued for each operation of the image analysis model. The ID number is included in each model operation record in the log, and the ID number is also included in each detailed record. A specific detailed record is associated with each model operation record through the ID number. The link information is included in the detailed record, and the analysis target image is associated with the detailed record based on the link information.

In a case where the user clicks on a specific model operation record in the displayed log, the report creation unit 42 acquires a specific detailed record associated with the specific model operation record and acquires a specific analysis target image based on the link information included in the specific detailed record.

Then, the report creation unit 42 creates the model operation report based on information included in the specific model operation record, information included in the acquired detailed record, and the acquired analysis target image. The model operation report is displayed on the display 26. The model operation report includes the analysis target image as input information and the analysis result as output information, as will be described below. By referring to the information, the user evaluates the performance or the state of the image analysis model.

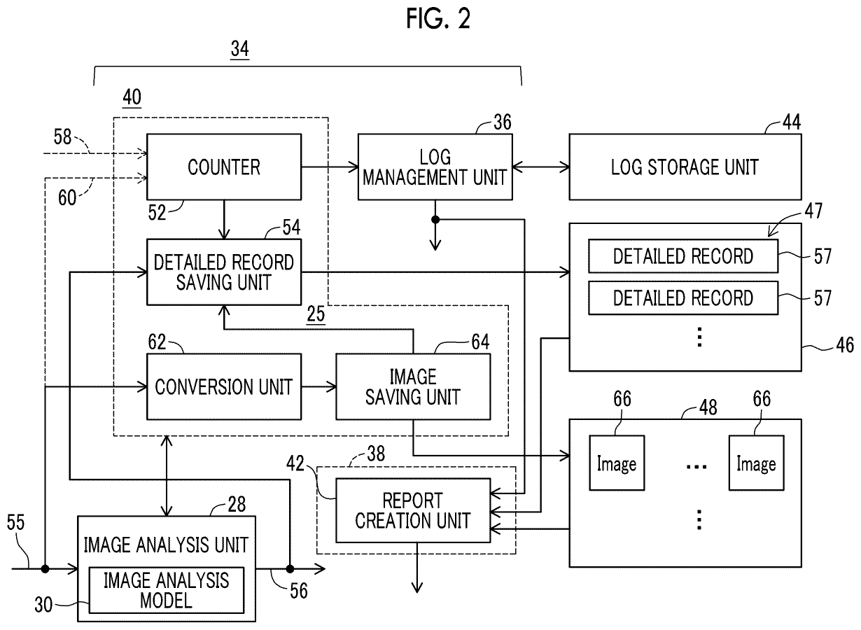

FIG. 2 shows a configuration example of the model operation management unit 40. In FIG. 2, the same reference numerals are designated by the same components as those shown in FIG. 1. The contents shown in FIG. 2 correspond to an algorithm of the model operation verification method according to the embodiment.

The information processing unit 34 includes the log management unit 36, the log analysis unit 38, and the model operation management unit 40. The log is constructed on the log storage unit 44. The model operation management unit 40 includes a counter 52, a detailed record saving unit 54, a conversion unit 62, and an image saving unit 64. The detailed record saving unit 54 and the image saving unit 64 function as detailed information saving means 25. The image analysis unit 28 includes the image analysis model 30. In a case where a tomographic image 55 is input to the image analysis model 30, the tomographic image is analyzed by the image analysis model 30, and an analysis result 56 is output from the image analysis model 30.

The counter 52 issues an AI processing request identifier (hereinafter, referred to as an ID number) for each operation of the image analysis model 30. In a case where the image analysis model 30 performs repeated operations, the counter 52 performs a count-up operation, and the ID number is sequentially increased. For example, the counter 52 may issue the ID number in response to a processing request from a main controller (refer to a reference numeral 58), or the counter 52 may issue the ID number in response to an input event of the tomographic image 55 (refer to a reference numeral 60). The issued ID number is sent to the log management unit 36 and the detailed record saving unit 54.

The log management unit 36 includes the ID number in the model operation record in a case of creating the model operation record. The detailed record saving unit 54 includes the ID number in the detailed record in a case of creating the detailed record. The ID number issued by the counter 52 may be sent to the log management unit 36 and the detailed record saving unit 54 through the image analysis unit 28. The analysis result of the image analysis model 30 is also sent to the detailed record saving unit 54.

The conversion unit 62 applies low-resolution conversion to the tomographic image 55, thereby generating a low-resolution image. Examples of the low-resolution image include a thumbnail image. The low-resolution conversion is processing of reducing the amount of information. Examples of a reduction rate include ratios such as 1/5, 1/10, or less than 1/10. Pixel thinning-out processing, compression processing, or the like may be applied. From the viewpoint of verification of the image analysis model 30, both the tomographic image 55 and the low-resolution image can be referred to as the analysis target images. Hereinafter, the low-resolution image will be referred to as an analysis target image.

The image saving unit 64 saves an analysis target image 66 in the analysis target image storage unit 48. The analysis target image storage unit 48 stores the number of analysis target images 66 corresponding to the number of operations of the image analysis model 30. A saving deadline may be set for each individual analysis target image 66, and the analysis target image 66 may be erased upon the expiration of the saving deadline. Alternatively, the entire storage content of the analysis target image 66 may be cleared in response to an explicit instruction from the user. The image saving unit 64 notifies the detailed record saving unit 54 of information indicating the location of each analysis target image 66 as the link information.

The detailed record saving unit 54 creates the detailed record including the ID number, the link information, and the analysis result and stores the detailed record in the detailed record storage unit 46. The detailed record storage unit 46 stores a detailed record column 47. The detailed record column 47 consists of a plurality of detailed records 57.

In a case where a specific model operation record in the log is selected by the user, the report creation unit 42 in the log analysis unit 38 acquires, based on the ID number included in the specific model operation record, a specific detailed record having the same ID number from the detailed record storage unit 46 and acquires, based on the link information included in the specific detailed record, a specific analysis target image corresponding to the specific detailed record from the analysis target image storage unit 48. Actually, the model operation record includes a portion embedded with a hyperlink for specifying the location of the detailed record. By clicking on that portion, the report creation unit 42 immediately acquires the specific detailed record.

As already described, the report creation unit 42 creates the model operation report including the information included in the specific model operation record, the information included in the specific detailed record, and the analysis target image. The model operation report is displayed on the display.

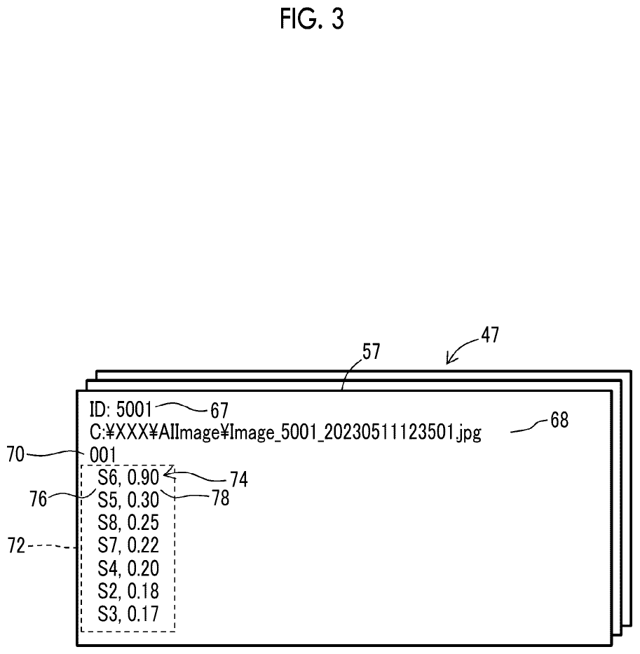

FIG. 3 shows the detailed record column 47. The detailed record column 47 consists of the plurality of detailed records 57 corresponding to a plurality of operations of the image analysis model. Each detailed record 57 includes an ID number 67, link information (URL) 68, an identifier 70 of the image analysis model, and an analysis result 72. The analysis result 72 is composed of a plurality of items 74 corresponding to the plurality of classes. Each item 74 is composed of a class identifier 76 and a score 78. The class identifier 76 indicates the type of the tissue cross section to be identified. The score 78 is a confidence level. The content of the analysis result 72 may be changed depending on the image analysis model to be used.

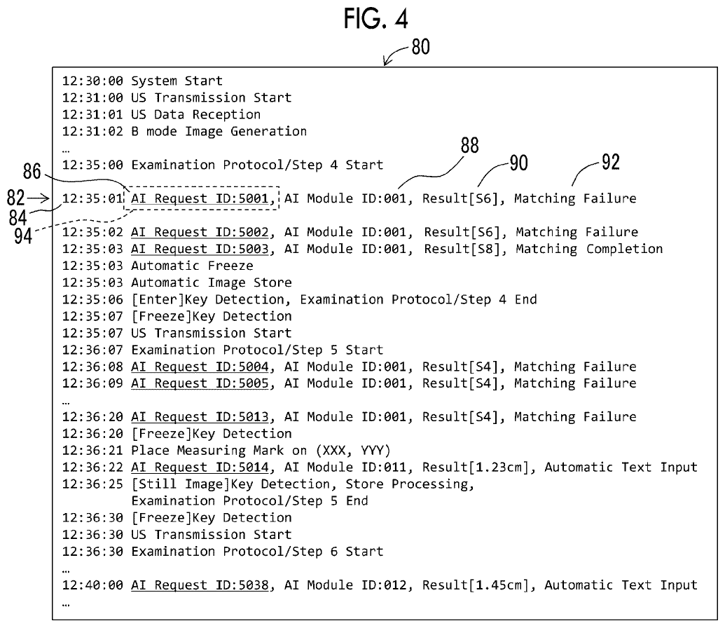

FIG. 4 shows an example of the log. A log 80 reflects, for example, a process of executing a plurality of steps constituting an examination protocol. The log 80 is composed of a plurality of records (rows) arranged in time series order. Basically, one record is generated for one operation of the ultrasound diagnostic apparatus (including an event, a state change, and the like). The log 80 includes a plurality of model operation records.

For example, a model operation record 82 includes an execution time 84, an ID number 86, an image analysis model identifier 88, a representative analysis result 90, analysis success/failure indication information 92, and the like. The URL of the detailed record is embedded in a portion (a portion displayed in a predetermined color with an underline) 94 corresponding to the ID number as hyperlink information. By clicking on the portion 94, the corresponding detailed record is instantly specified, and then the model operation report is created based on the detailed record, and the model operation report is displayed in a pop-up manner. For example, the model operation report is superimposed and displayed on the log.

In the displayed plurality of records, it is possible to easily specify each individual model operation record by using the underline, the ID number, or the like as a clue. A special label may be added to each individual model operation record in order to enhance the identifiability or visibility of each individual model operation record.

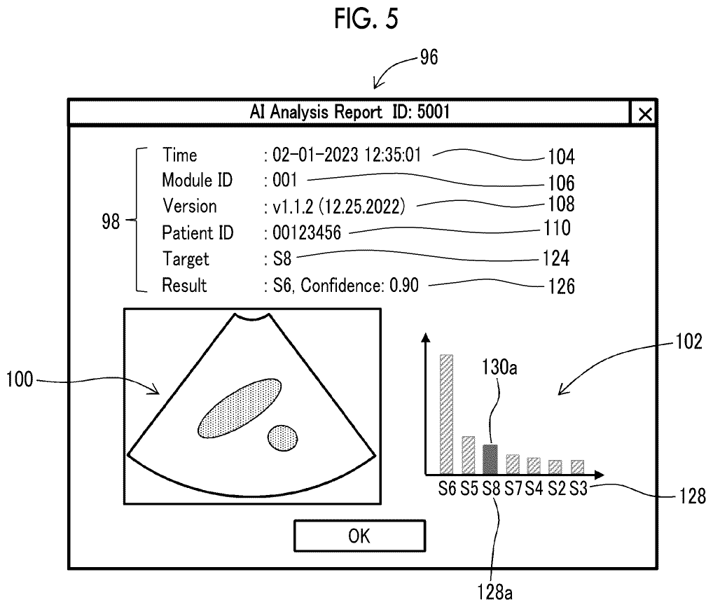

FIG. 5 shows a first example of the model operation report. A model operation report 96 includes text information 98, an analysis target image 100, and a graph 102. The text information 98 includes an execution time 104, an identifier 106 of the image analysis model, a version 108 of the image analysis model, a subject ID 110, a type 124 of the tissue cross section to be observed, and a representative analysis result 126.

The analysis target image 100 is the low-resolution image. In the shown example, a still image is displayed, but a moving image may be displayed. The graph 102 is created based on a plurality of scores (a plurality of confidence levels) constituting the analysis result. The plurality of scores are arranged from left to right in descending order, and the magnitude of each score is represented by the height of each bar. A plurality of classes (tissue cross section types) corresponding to the plurality of scores are displayed along a horizontal axis (refer to a reference numeral 128).

In the shown example, the type (class) of the tissue cross section to be observed is S8 (refer to a reference numeral 128a), and the score corresponding to S8 is relatively low (130a). The graph 102 indicates that the acquisition of the target tomographic image has failed (and is not completed). Examples of the cause include the quality of the analysis target image and the performance of the image analysis model. In a case where the content of the tomographic image is not appropriate because the position and the orientation of the probe are not appropriate, the score of the class of interest is naturally low. On the other hand, in a case where the content of the tomographic image is appropriate, but the score of the class of interest is low, it may indicate that the performance of the image analysis model is not sufficient.

In this way, by collating the input information and the output information included in the model operation report, it is possible to comprehensively verify the operation of the image analysis model. The content of the model operation report may be sequentially switched along a time axis. The model operation report is displayed as necessary. That is, in a case of simply confirming a series of operations of the image analysis model, the log itself need only be referred to.

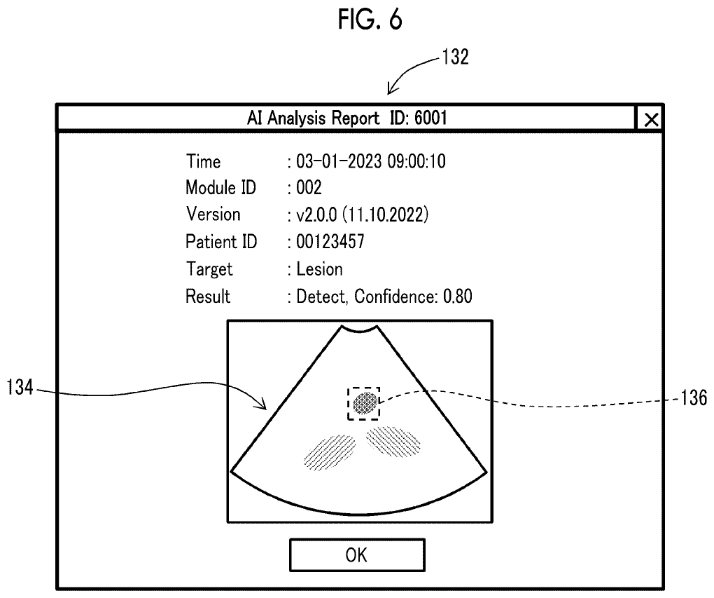

FIG. 6 shows a second example of the model operation report. For example, in a case where an image analysis model that detects the lesion part is used, a model operation report 132 shown in FIG. 6 is displayed. A box 136 surrounding the detected lesion part is shown in an analysis target image 134. The box 136 is a graphic element displayed together with the tomographic image. By referring to the display coordinates and the size of the box 136, it is possible to verify whether or not the image analysis model has correctly detected the lesion part. In this case as well, the moving image may be displayed instead of the still image.

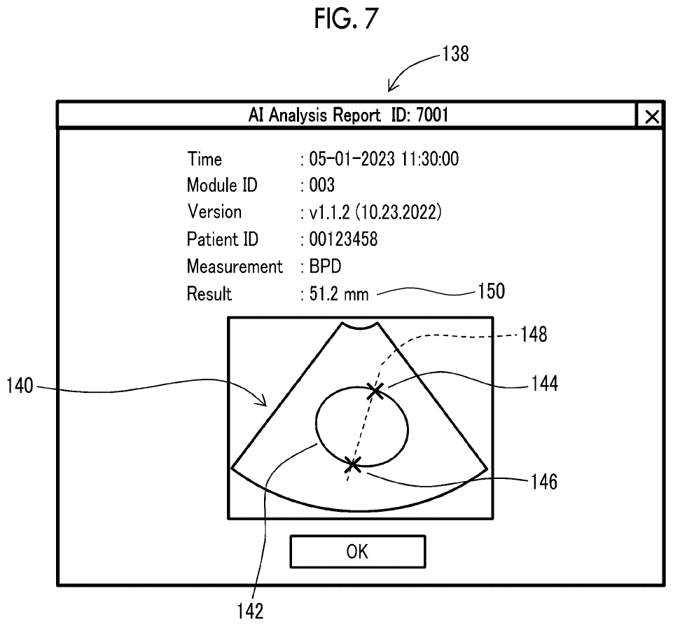

FIG. 7 shows a third example of the model operation report. For example, in a case where an image analysis model that measures a tissue is used, a model operation report 138 shown in FIG. 7 is displayed. An analysis target image 140 includes a tissue image 142 that is a measurement target. Two positions of interest in the tissue image 142 are automatically specified, and two markers 144 and 146 are displayed at the two positions of interest. An auxiliary line 148 passing through these two positions of interest is also displayed. A distance between the two positions of interest is automatically measured on the auxiliary line 148. A result of the measurement is displayed as a numerical value (refer to a reference numeral 150). For example, by comparing the tissue image 142 with the positions of the two markers 144 and 146, it is possible to verify whether or not the operation of the image analysis model is appropriate.

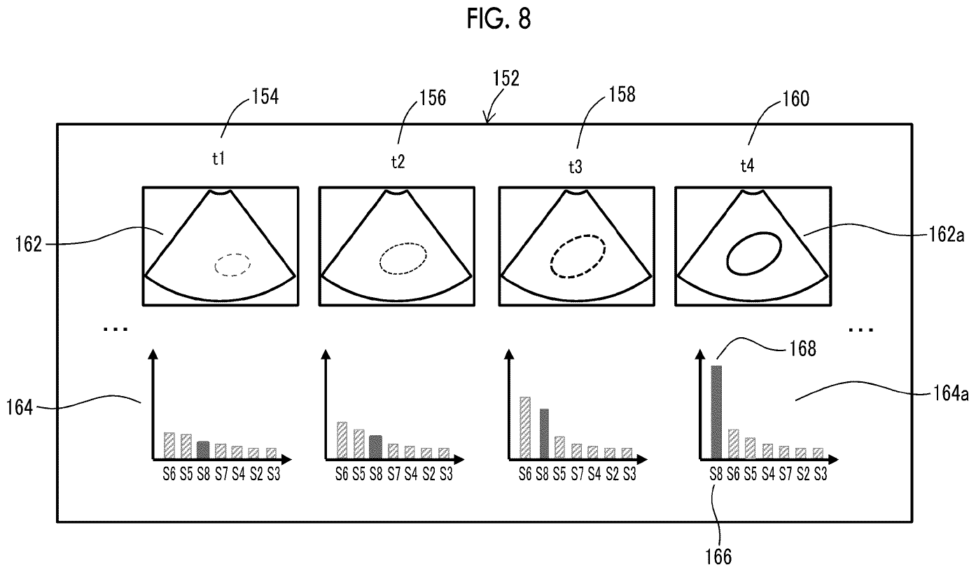

FIG. 8 shows a fourth example of the model operation report. The image analysis model is a model for class classification. Seven scores corresponding to seven tissue cross sections from S2 to S8 are calculated. Each score indicates a probability that an observation cross section corresponds to a specific tissue cross section.

A shown model operation report 152 includes a plurality of information sets 154 to 160 arranged in time series order (refer to t1 to t4). That is, the model operation report 152 corresponds to the plurality of model operation records, in other words, the model operation report 152 is created based on the plurality of detailed records.

Each of the information sets 154 to 160 is composed of an analysis target image 162 as the input information and a graph 164 showing the analysis result as the output information. Each graph 164 includes a plurality of bars representing the plurality of scores. The bar filled with a dark color is a bar corresponding to the tissue cross section (S8) to be observed. In t4, an analysis target image 162a is input to the image analysis model, and a satisfactory analysis result is obtained by the analysis. That is, in a graph 164a, a large value is calculated as the score corresponding to the tissue cross section (S8) to be observed. The height of a bar 168 indicating the score is large, and the bar 168 is located at the forefront. By displaying such a model operation report, it is possible to intuitively and easily understand the temporal change in the operation of the image analysis model.

As described above, according to the above-described embodiment, it is possible to verify the performance or the state of the image analysis model in detail by referring to the detailed information (the input information and the output information) included in the model operation report. The ultrasound image other than the tomographic image may be analyzed. Operations of a plurality of image analysis models that operate in parallel or selectively may be recorded.

What is claimed is:

1. An ultrasound diagnostic apparatus comprising:
an analysis unit that includes an image analysis model which has been trained through machine learning to analyze an ultrasound image;
a log creation unit that creates a log consisting of a plurality of operation records by recording a plurality of operations including an operation of the image analysis model in time series order;
a saving unit that saves detailed information for model operation verification for each operation of the image analysis model; and
a report creation unit that creates, in a case where a specific model operation record in the log is selected, a model operation report including detailed information associated with the specific model operation record.

2. The ultrasound diagnostic apparatus according to claim 1,
wherein the saving unit saves an analysis target image as the detailed information for each operation of the image analysis model,
the analysis target image is an ultrasound image input to the image analysis model or an image corresponding to the ultrasound image, and
the model operation report includes the analysis target image.

3. The ultrasound diagnostic apparatus according to claim 2, further comprising:
a conversion unit that converts the ultrasound image input to the image analysis model into a low-resolution image,
wherein the analysis target image is the low-resolution image.

4. The ultrasound diagnostic apparatus according to claim 1,
wherein the saving unit saves an analysis result of the image analysis model as the detailed information for each operation of the image analysis model, and
the model operation report includes the analysis result.

5. The ultrasound diagnostic apparatus according to claim 4,
wherein the analysis result includes a plurality of scores corresponding to a plurality of classes,
the report creation unit creates a graph based on the plurality of scores, and
the model operation report includes the graph.

6. The ultrasound diagnostic apparatus according to claim 1,
wherein the saving unit includes
an image saving unit that saves an analysis target image for each operation of the image analysis model, and
a record saving unit that saves a detailed record including an analysis result of the image analysis model for each operation of the image analysis model,
the analysis target image is a low-resolution image generated from an ultrasound image input to the image analysis model, and
the detailed information in the model operation report includes the analysis target image, which is input information of the image analysis model, and the analysis result, which is output information of the image analysis model.

7. The ultrasound diagnostic apparatus according to claim 1,
wherein each model operation record included in the log includes a portion embedded with a hyperlink for specifying a location of the detailed information associated with the model operation record, and
the model operation report is displayed upon selection of the portion.

8. A model operation verification method comprising:
a step of creating a log consisting of a plurality of operation records by recording, in time series order, a plurality of operations including an operation of an image analysis model that has been trained through machine learning to analyze an ultrasound image;
a step of saving detailed information for model operation verification for each operation of the image analysis model;
a step of displaying the log;
a step of accepting selection of a specific model operation record in the displayed log;
a step of specifying detailed information associated with the specific model operation record;
a step of creating a model operation report including the detailed information; and
a step of displaying the model operation report.

9. A non-transitory storage medium storing a program for causing an ultrasound diagnostic apparatus to execute a model operation verification method, the model operation verification method including:
a step of creating a log consisting of a plurality of operation records by recording, in time series order, a plurality of operations including an operation of an image analysis model that has been trained through machine learning to analyze an ultrasound image;
a step of saving detailed information for model operation verification for each operation of the image analysis model;
a step of displaying the log;
a step of accepting selection of a specific model operation record in the displayed log;
a step of specifying detailed information associated with the specific model operation record;
a step of creating a model operation report including the detailed information; and
a step of displaying the model operation report.
